(19) United States
(12) Patent Application Publication
(10) Pub. No.: US 2018/0372732 A1
(43) Pub. Date: Dec. 27, 2018

(54) Method for Detecting Particles in a Sample, Detection Device, and Microfluidic System for Examining a Sample

(71) Applicant: Robert Bosch GmbH, Stuttgart (DE)

(72) Inventors: Thomas BRETTSCHNEIDER, Leonberg (DE); Jochen HOFFMANN, Renningen (DE)

(21) Appl. No.: 16/062,110

(22) PCT Filed: Dec. 16, 2016
(86) PCT No.: PCT/EP2016/081470

§ 371 Date: Jun. 14, 2018

(30) Foreign Application Priority Data
Dec. 18, 2015 (DE) 10 2015 225 849.1

(51) Int. Cl. G01N 33/543; G01N 15/10; G01N 27/74; G01R 33/12

(52) U.S. Cl. CPC G01N 33/54326; G01R 33/1269; G01N 27/745; G01N 15/1031

(57) ABSTRACT

A method for detecting particles in a sample includes adding magnetic detection particles to the sample, the magnetic detection particles configured to bind to the particles for detection, and reading in a measurement signal from an interface to at least one magnetic field sensor. The measurement signal includes at least one acquired characteristic of a magnetic field extending at least through a partial quantity of the sample. The method further includes ascertaining a fluctuation intensity of the at least one characteristic of the magnetic field, the fluctuation intensity dependent on the detection particles. The method also further includes determining a state of binding of at least one detection particle as unbound or bound to a particle for detection based on the determined fluctuation intensity in order to detect the particles in the sample.

### PRIOR ART

The invention relates to a device or a method according to the preamble of the independent claims. The present invention also relates to a computer program.

It is for example known for a diffusion constant of a particle in Brownian motion to be determined from the trajectory thereof by means of an optical apparatus.

### DISCLOSURE OF THE INVENTION

Against this background, with the approach proposed here, a method, furthermore a device that uses said method and finally a corresponding computer program are presented, as per the main claims. Advantageous refinements and improvements of the device specified in the independent claim are possible by means of the measures specified in the dependent claims.

In embodiments of the present invention, particles for detection in a sample can be detected in particular through the magnetic acquisition of magnetic detection particles that can form a bind with the particles for detection. Here, it is possible in particular to identify a state of binding of the detection particles, which can be set in relation to a fluctuation of the acquired magnetic field.

According to embodiments of the present invention, it is advantageously possible in particular for a process time or duration for the analysis of samples or for the detection of particles to be shortened. It is also possible to realize a qualitative and additionally or alternatively a quantitative detection of particles. Furthermore, it is for example necessary for only at least one detection particle to be added to a sample, such that material costs can also be reduced. Furthermore, a capacity for integration into so-called pocket laboratory systems or lab-on-chip systems can be simplified. According to embodiments of the present invention, it can also be made possible to detect individual particles. According to embodiments of the present invention, detection or analysis can be performed in contactless fashion and without optically transparent access to the sample. Furthermore, a material selection from electrical non-conductors for such lab-on-chip systems can be enlarged.

A method for detecting particles in a sample is proposed, wherein the method has the following features:

reading in a measurement signal, which represents at least one acquired characteristic of a magnetic field extending at least through a partial quantity of the sample, from an interface to at least one magnetic field sensor, wherein magnetic detection particles are added to the sample, which magnetic detection particles are designed to bind to the particles for detection;

ascertaining a fluctuation intensity, which is dependent on the detection particles, of the at least one characteristic of the magnetic field; and

determining a state of binding of at least one detection particle as unbound or bound to a particle for detection in a manner dependent on the determined fluctuation intensity in order to detect the particles in the sample.

This method may be implemented for example in software or hardware, or in a mixed form of software and hardware, for example in a device or a control unit. The sample may be a liquid sample or a sample in a liquid state of aggregation. Here, the sample may be arranged in a vessel and optionally agitated. Here, the vessel may be formed as a container or as a channel, wherein the sample may optionally be moved through the vessel. The fluctuation intensity may represent a range of variation of the measurement signal.

For example, by means of at least one magnetic field sensor, it is possible to identify whether a magnetic particle is bound to a particle for detection, in particular a biological particle such as for example a cell, without performing a complex immunoassay workflow, for example. A method for detecting particles in the form of cells may be used for microfluidic lab-on-chip systems for medical diagnosis.

In one embodiment, in the step of determining, the state of binding is determined as unbound if the fluctuation intensity lies in a first value range. Here, in the step of determining, the state of binding may be determined as bound to a particle for detection if the fluctuation intensity lies in a second value range which represents lower fluctuation intensities than the first value range. The fluctuation intensity of an unbound detection particle may be greater than the fluctuation intensity of a detection particle bound to a particle for detection.

Such an embodiment offers the advantage that the state of binding can be reliably determined, and therefore more accurate and reliable detection of a presence or absence of particles for detection is made possible.

Also, in the step of reading in, a measurement signal may be read in which represents an intensity of the magnetic field and additionally or alternatively a direction of the magnetic field versus the time. Such an embodiment offers the advantage that an accuracy of an acquisition of the at least one magnetic detection particle, or of a changing magnetic field effected thereby, can be increased.

Furthermore, the method may have a step of adding a predefined quantity of the magnetic detection particles to the sample. Here, the detection particles may be contained in a liquid, solution or the like. Such an embodiment offers the advantage that a concentration of the detection particles in the sample can be exactly set. It is also possible here, for example, for the concentration of the detection particles in the sample to be set such that in each case one detection particle is situated in the region of the magnetic field sensor, in order to increase a measurement accuracy.

Here, in the step of adding, detection particles may be added which are designed to bind to antibodies and additionally or alternatively to surface proteins of the particles for detection. Here, the particles for detection are biological particles. It is additionally or alternatively possible, in the step of adding, for magnetic detection particles to be added to the sample. It is thus possible, according to one embodiment, for the detection particles to have a magnetic section and a binding section, in particular an antibody section or the like. Such an embodiment offers the advantage that, for example, cells or microorganisms can be specifically and reliably detected. In the case of paramagnetic detection particles, an acquisition accuracy can be further increased, wherein furthermore, in particular, a calibration of the at least one magnetic field sensor and of the detection method can be permitted or facilitated.

Furthermore, the method may have a step of generating the magnetic field. Here, the measurement signal read in in the step of reading in may represent at least one characteristic of the generated magnetic field. Such an embodiment offers the advantage that fluctuations of the generated magnetic field effected by the at least one detection particle can be registered reliably and accurately.

Here, in the step of generating, a homogeneous magnetic field may be generated as the magnetic field. In addition or alternatively, in the step of generating, a magnetic field may be generated which is designed to immobilize at least one detection particle adjacent to the at least one magnetic field sensor. Such an embodiment offers the advantage that, by means of a homogeneous magnetic field, a rotation of the detection particles can be prevented, and thus an accuracy of detection can be increased, wherein, by virtue of the detection particles being magnetically held stationary even in a flowing sample, an accurate detection of individual detection particles can be performed. Furthermore, by means of the immobilization, a detection particle can be positioned exactly relative to a magnetic field sensor.

The method may also have a step of circulating the sample with the added detection particles. Here, the step of circulating may be capable of being performed before, during and additionally or alternatively after an acquisition of the at least one characteristic of the magnetic field. Here, the sample may be circulated using a pump or an agitator. In particular, the sample may be circulated between a determination of the state of binding of a first detection particle and a determination of the state of binding of a second detection particle. Alternatively, the sample may be circulated continuously, wherein time intervals between the determination of the state of binding of different detection particles can be shortened, and low-frequency signal components can be filtered out of the measurement signal. Such an embodiment offers the advantage that the state of binding of detection particles can be determined in succession, and additionally or alternatively an overall duration of the detection can be shortened.

A detection device is also proposed which is designed to perform steps of an embodiment of the above-stated method in corresponding units.

The approach proposed here thus furthermore provides a detection device or device which is designed to carry out, actuate or implement the steps of a variant of a method proposed here in corresponding means. It is also possible by means of this design variant of the invention, in the form of a device, for the object on which the invention is based to be achieved quickly and efficiently.

For this purpose, the device may have at least one processing unit for processing signals or data, at least one memory unit for storing signals or data, at least one interface to a sensor or an actuator for reading in sensor signals from the sensor or for outputting data or control signals to the actuator, and/or at least one communication interface for reading in or outputting data that are embedded into a communication protocol. The processing unit may for example be a signal processor, a microcontroller or the like, wherein the memory unit may be a flash memory, an EPROM or a magnetic memory unit. The communication interface may be designed to read in or output data wirelessly and/or in wired fashion, wherein a communication interface that can read in or output data in wired fashion may for example read in said data from a corresponding data transmission line, or output said data into a corresponding data transmission line, in electrical or optical fashion.

A device may in the present case be understood to mean an electrical unit which processes sensor signals and, in a manner dependent thereon, outputs control and/or data signals. The device may have an interface which may be formed by hardware and/or software. In a hardware embodiment, the interfaces may for example be part of a so-called system ASIC which encompasses a wide variety of functions of the device. It is however also possible for the interfaces to be dedicated integrated circuits or to be composed at least partially of discrete components. In a software embodiment, the interfaces may be software modules which are present for example on a microcontroller in addition to other software modules.

In one advantageous embodiment, control of at least one magnetic field sensor and additionally or alternatively of at least a subsection of a microfluidic system is performed by means of the detection device or device. For this purpose, the device may for example access sensor signals such as the measurement signals. The device may be designed to generate and make available or output at least one actuation signal in response to the measurement signal, in response to the ascertained fluctuation intensity and additionally or alternatively in response to the determined state of binding. The actuation is performed by means of actuators such as pumps, agitators and additionally or alternatively means for generating a magnetic field.

Also proposed is a microfluidic system for analyzing a sample, which microfluidic system has the following features:

an embodiment of the detection device mentioned above; and

an interface for coupling to the at least one magnetic field sensor, wherein the measurement signal can be transmitted via the interface to the detection device.

In conjunction with the microfluidic system, the detection device according to one of the embodiments mentioned above can be advantageously used for detecting the particles during the analysis of the sample. The interface may be designed to produce a releasable mechanical connection, which is additionally or alternatively capable of transmitting signals, between the microfluidic system and the at least one magnetic field sensor.

In one embodiment, the microfluidic system may have a microfluidic channel in which the sample may be conductable. Here, the detection device and additionally or alternatively the interface may be arranged adjacent to the microfluidic channel. In particular, the microfluidic system may be designed as a so-called pocket laboratory (LoC, Lab on Chip; laboratory on a chip). Such an embodiment offers the advantage that the analysis of the sample with detection of the particles can be performed inexpensively, in a space-saving manner and reproducibly.

Here, the interface may be designed to arrange the at least one magnetic field sensor in contact with, or move the at least one magnetic field sensor into contact with, a wall of the microfluidic channel. Here, the wall of the microfluidic channel may be elastic, wherein the interface may be designed to deform the wall of the microfluidic channel by means of the at least one magnetic field sensor. Such an embodiment offers the advantage that an acquisition accuracy can be further increased, because a spacing between the sample and the at least one magnetic field sensor can be minimized.

The microfluidic system may also have at least one magnetic field sensor, which may be removably coupled or couplable to the interface. Here, the at least one magnetic field sensor may be designed for example as a one-dimensional magnetic field sensor or as a three-dimensional magnetic field sensor. Such an embodiment offers the advantage that firstly an accurate detection of particles can be achieved using simple and inexpensive means using a one-dimensional sensor, and secondly certainty in the detection of the state of binding can be further increased through the use of a three-dimensional sensor.

Also advantageous is a computer program product computer program having program code which can be stored on a machine-readable carrier or memory medium such as a semiconductor memory, a hard drive memory or an optical memory and which is used for carrying out, implementing and/or actuating the steps of the method according to one of the embodiments described above, in particular if the program product or program is executed on a computer or a device.

Exemplary embodiments of the invention are illustrated in the drawings and are discussed in more detail in the following description, in which:

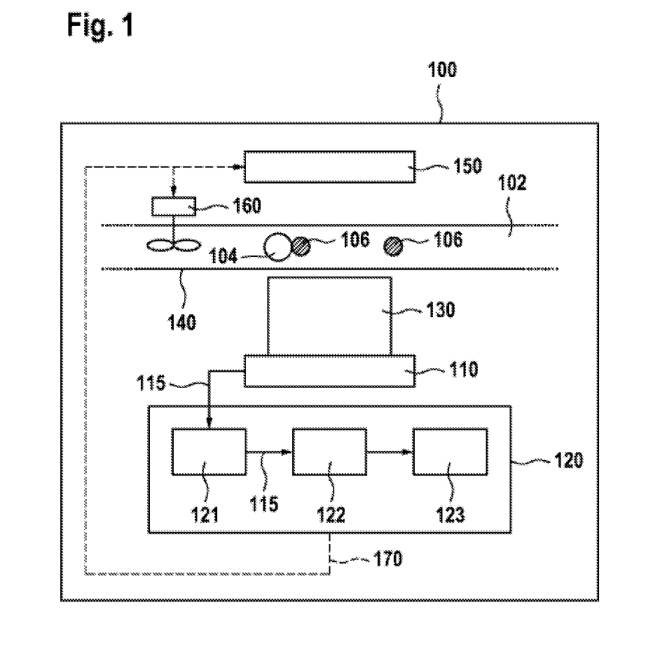

FIG. 1 is a schematic illustration of a microfluidic system according to an exemplary embodiment;

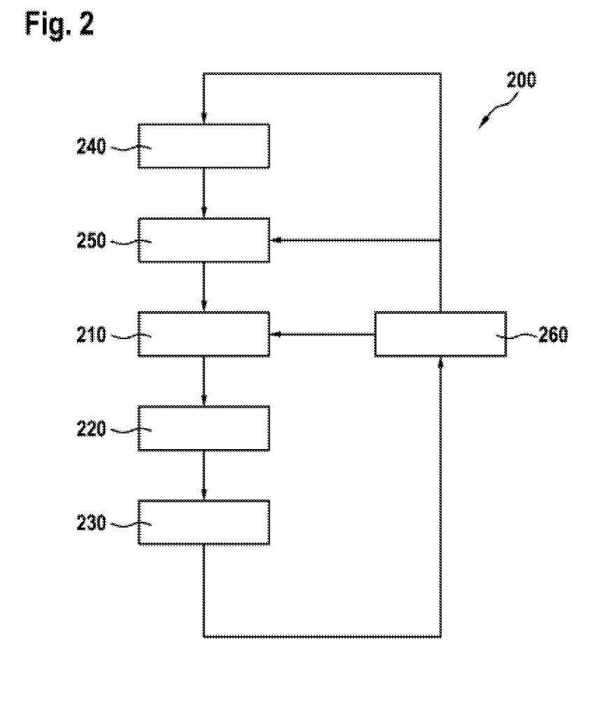

FIG. 2 shows a flow diagram of a method for detecting particles as per an exemplary embodiment;

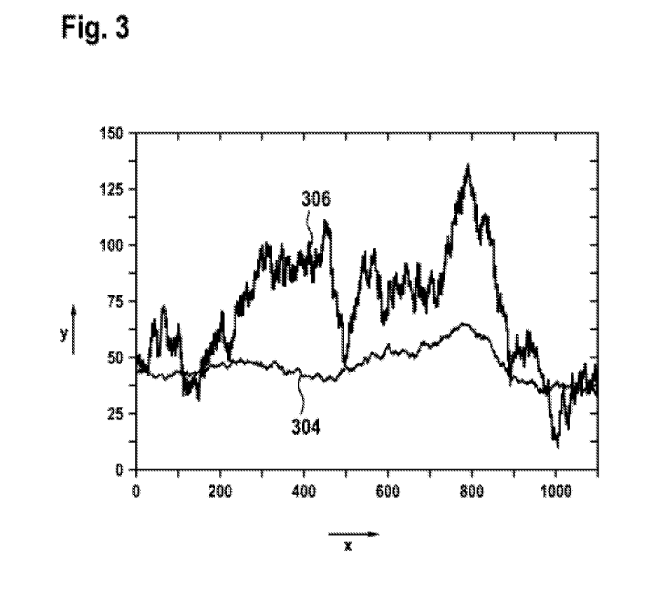

FIG. 3 shows a diagram of magnetic field intensities versus the time;

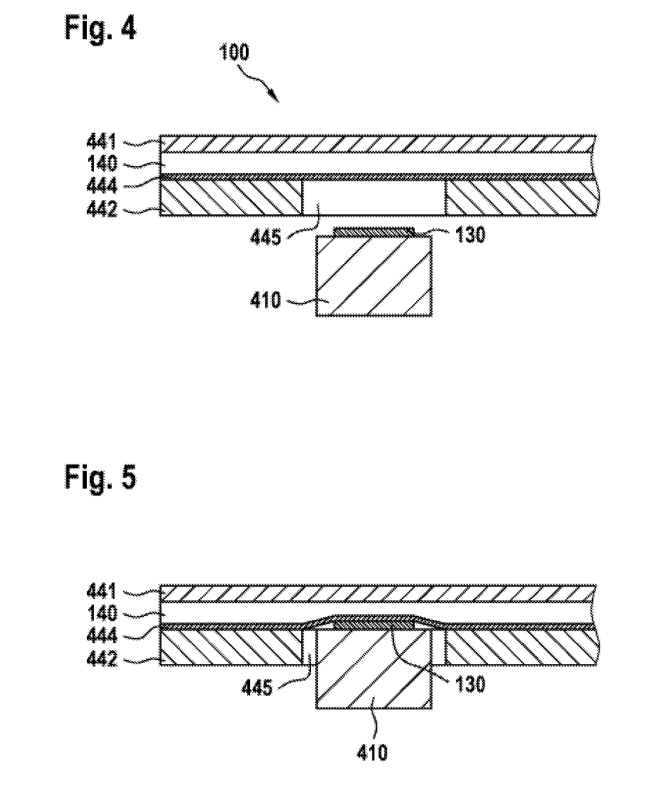

FIG. 4 is a schematic sectional illustration of a part of a microfluidic system as per an exemplary embodiment; and

FIG. 5 is a schematic sectional illustration of a part of a microfluidic system as per an exemplary embodiment.

In the following description of expedient exemplary embodiments of the present invention, identical or similar reference designations will be used for the elements of similar action illustrated in the various figures, wherein a repeated description of these elements will be omitted.

Fundamental principles for exemplary embodiments and backgrounds to the analysis of samples and to the detection of particles will firstly be discussed. In analytical chemistry, use is for example made of so-called bioassays in order to detect substances such as proteins, DNA molecules (DNA, deoxyribonucleic acid) or cells, for example pathogens, body cells or circulating tumor cells (CTCs), for example for diagnostic purposes. For example, so-called immunoassays are commonly used for detecting cells. Here, detection antibodies are used which bind to a particular surface protein of a cell. To identify the cells bound by the detection antibodies, use is made of fluorescing probes or magnetic particles, so-called identification antibodies, which bind either to the detection antibodies or to another surface protein of the cell. If the detection antibody has been immobilized on a surface, the identification antibodies and thus also the cells for detection can be localized and detected; this is a so-called sandwich immunoassay. Immunoassays exist in a wide variety of modifications of this. Conventional immunoassays involve for example a sequence of numerous substeps, for example addition of sample, washing (removal of excess), addition of detection antibodies, washing, addition of identification antibodies with probe, washing, detection. Such immunoassays can be complex to perform. The end result is information regarding whether or not the probes (for example magnetic particles) have bound to the cells for detection. Furthermore, it is optionally possible to determine how many probes have bound to cells in order to permit a determination of concentration.

FIG. 1 is a schematic illustration of a microfluidic system 100 for analyzing a sample 102 as per an exemplary embodiment. The sample 102 contains particles 104 for detection. Furthermore, magnetic detection particles 106 have been added to the sample 102. The magnetic detection particles are designed to bind to the particles 104 for detection, or to form a chemical, physical or physicochemical bond to the particles 104 for detection.

The microfluidic system 100 has an interface 110 and a detection device 120. The interface 110 is designed to permit a coupling to at least one magnetic field sensor 130. In other words, the interface 110 is designed to be couplable to at least one magnetic field sensor 130. The coupling between the interface 110 and the at least one magnetic field sensor 130 is in this case mechanical and/or capable of transmitting signals. By the interface 110, a measurement signal 115 can be transmitted from a magnetic field sensor 130 coupled to the interface 110 to the detection device 120.

The illustration of FIG. 1 shows a magnetic field sensor 130 coupled to the interface 110. Here, the magnetic field sensor 130 is removably coupled to the interface 110. The magnetic field sensor 130 is designed to acquire at least one characteristic of a magnetic field that extends through at least a partial quantity of the sample 102. The at least one characteristic of the magnetic field is in this case in particular an intensity and/or a direction of the magnetic field versus the time. Furthermore, the magnetic field sensor 130 is designed to make available, or output, the measurement signal 115. The measurement signal 115 represents the at least one acquired characteristic of the magnetic field.

The detection device 120 is designed to detect the particles 104 for detection, or the particles 104 in the sample 102. For this purpose, the detection device 120 has a reading-in means 121, an ascertainment means 122 and a determination means 123. The detection device 120 is designed to detect, in response to the measurement signal 115 and/or using the measurement signal 115, whether or not the particles 104 are present in the sample 102.

The reading-in means 121 of the detection device 120 is designed to read in the measurement signal 115 from the interface 110 to the magnetic field sensor 130. Furthermore, the reading-in means 121 is designed to output the read-in measurement signal 115 to the ascertainment means 122 or make the read-in measurement signal 115 available to the ascertainment means 122. The reading-in means 121 is connected with signal transmitting capability to the ascertainment means 122.

The ascertainment means 122 is designed to ascertain, using the measurement signal 115, a fluctuation intensity of the at least one characteristic of the magnetic field represented by the measurement signal 115. The fluctuation intensity is in this case dependent on the detection particles 106. In other words, the fluctuation intensity of the measurement signal 115 or of the at least one characteristic, represented by the measurement signal 115, of the magnetic field is dependent on a presence of at least one detection particle 106 in the magnetic field.

The determination means 123 is connected with signal transmitting capability to the ascertainment means 122. The determination means 120 is designed to determine, in a manner dependent on the fluctuation intensity ascertained by the ascertainment means 122, a state of binding of at least one detection particle 106 in the magnetic field as unbound or bound to a particle 104 for detection. On the basis of the determined state of binding of the at least one detection particle 106, the particles 104 in the sample 102 can be detected qualitatively and/or quantitatively.

For example, the determination means 123 is designed to determine the state of binding as unbound if the ascertained fluctuation intensity lies in a first value range. Furthermore, the determination means 123 is designed to determine the state of binding as bound to a particle 104 for detection if the ascertained fluctuation intensities lies in a second value range. Here, the second value range represents lower fluctuation intensity than the first value range. Therefore, a relatively low fluctuation intensity or a fluctuation intensity in the first value range corresponds to a bound state of the detection particle 106, wherein a relatively high fluctuation intensity or a fluctuation intensity in the first value range corresponds to an unbound state of the detection particle 106.

In the exemplary embodiment shown in FIG. 1, the microfluidic system 100 furthermore has a microfluidic channel 140. The microfluidic channel 140 is designed to conduct the sample 102 past the magnetic field sensor 130. Therefore, in the illustration in FIG. 1, the sample 102 is contained or arranged in the microfluidic channel 140.

In the exemplary embodiment illustrated in FIG. 1, the interface 110 with the coupled-on magnetic field sensor 130 is arranged adjacent to the sample 102 or to the microfluidic channel 140. In a further exemplary embodiment, the detection device 120 is additionally or alternatively arranged adjacent to the sample 102 or to the microfluidic channel 140. Optionally, the interface 110 is designed to arrange the magnetic field sensor 130 in contact with, or move the magnetic field sensor 130 into contact with, a wall of the microfluidic channel 140.

For example, the sample 102 comprises biological material, wherein the particles 104 for detection are biological particles, in particular cells, organic molecules or the like. Here, the detection particles 106 are designed to bind to antibodies and/or surface proteins of the particles 104 for detection.

In one exemplary embodiment, the detection particles 106 may additionally or alternatively be in the form of paramagnetic detection particles 106.

In the exemplary embodiment illustrated in FIG. 1, the microfluidic system 100 has a generating means 150 for generating the magnetic field. In particular, the generating means 150 is designed to generate a homogeneous magnetic field. In another exemplary embodiment, the generating means 150 is designed to generate a magnetic field in which at least one detection particle 106 can be immobilized adjacent to the magnetic field sensor 130.

Furthermore, the microfluidic system 100 as per in the exemplary embodiment shown in FIG. 1 has an optional circulating means 160. The circulating means 160 is designed to move or circulate the sample 102, in particular within the microfluidic channel 140. The circulating means 160 is designed for example as a pump or an agitator.

In the exemplary embodiment illustrated in FIG. 1, the detection device 120 is connected with signal transmitting capability to the generating means 150 and to the circulating means 160. Here, the detection device 120 is designed to output at least one actuation signal 170 to the generating means 150 and/or to the circulating means 160. Thus, the generating means 150 and/or the circulating means 160 are actuatable at least inter alia by means of the actuation signal 170 from the detection device 120.

FIG. 2 shows a flow diagram of a method 200 for detecting particles in a sample as per an exemplary embodiment. The method 200 for detecting can be implemented in conjunction with the detection device from FIG. 1 or a similar detection device. The method 200 for detecting can furthermore be implemented in conjunction with the microfluidic system from FIG. 1 or a similar microfluidic system.

The method 200 for detecting has a step 210 of reading in a measurement signal from an interface to at least one magnetic field sensor. Here, the measurement signal represents at least one acquired characteristic of a magnetic field extending through at least a partial quantity of the sample. Magnetic detection particles have been added to the sample, which magnetic detection particles are designed to bind to the particles for detection.

In the method 200, in a step 220 of ascertaining, which can be carried out following the step 210 of reading in, a fluctuation intensity, which is dependent on the detection particles, of the at least one characteristic of the magnetic field is ascertained. Furthermore, in a step 220 of determining, it is thereupon the case that, in a manner dependent on the fluctuation intensity ascertained in the step 220 of ascertaining, a state of binding of at least one detection particle is determined as unbound or bound to a particle for detection.

By carrying out the steps of the method 200, the particles in the sample can be qualitatively and/or quantitatively detected. Here, the step 210 of reading in, the step 220 of ascertaining and the step 230 of determining may be carried out in repeatable or repeated fashion, for example for a multiplicity of detection particles in succession.

In one exemplary embodiment, the method 200 for detecting has a step 240 of adding a predefined quantity of the magnetic detection particles to the sample. The step 240 of adding can in this case be performed before the step 210 of reading in. Here, in the step 240 of adding, detection particles may be added which are designed to bind to antibodies and/or surface proteins of the particles for detection, if the particles for detection are biological particles. In addition or alternatively, in the step 240 of adding, paramagnetic detection particles may be added to the sample.

In a further exemplary embodiment, the method 200 for detecting has a step 250 of generating, wherein the magnetic field is generated. The step 250 of generating can in this case be performed before the step 210 of reading in. Merely by way of example, the step 250 of generating can be performed between the step 240 of adding and the step 210 of reading in. Here, the measurement signal read in in the step 210 of reading in represents at least one characteristic of the magnetic field generated in the step 250 of generating. Here, it is optionally the case in the step 250 of generating that a homogeneous magnetic field is generated as the magnetic field. In addition or alternatively, in the step 250 of generating, a magnetic field is generated which is designed to immobilize at least one detection particle adjacent to the at least one magnetic field sensor.

In a yet further exemplary embodiment, the method 200 for detecting has a step 260 of circulating the sample with the added detection particles. The step 260 of circulating can be implemented for example by agitating or pumping the sample. Here, the step 260 of circulating can optionally be performed before the step 210 of reading in, in parallel with the step 210 of reading in, with the step 220 of ascertaining and with the step 230 of determining, after the step 230 of determining, and/or between the step 230 of determining and a subsequent execution of the step 210 of reading in.

FIG. 3 shows a diagram of magnetic field intensities versus the time. Here, on an abscissa axis labelled x of the diagram, the time is plotted, in particular in arbitrary units, wherein, on an ordinate axis labelled y of the diagram, measured magnetic field intensities are plotted, which are represented in measurement signals. The measurement signals are possible measurement signals which are generated, used and/or processed in the detection device from FIG. 1 or a similar detection device or in the microfluidic system from FIG. 1 or a similar microfluidic system.

A first graph 304 represents a first measurement signal with a first range of variation or fluctuation intensity. The first graph 304 represents a measurement signal for a detection particle bound to a particle for detection. Here, the first graph 304 has a low fluctuation intensity, that is to say the changes of the signal within one time step are small.

A second graph 306 represents a second measurement signal with a second range of variation or fluctuation intensity. The second graph 306 represents a measurement signal for a freely moving detection particle not bound to a particle for detection. Here, the second graph 306 exhibits a high fluctuation intensity, that is to say the changes of the signal within one time step are large or larger than in the case of the first graph 304.

Since particles move freely in a liquid, they travel along an erratic path owing to impacts against liquid molecules (Brownian motion). If a position of a particle versus the time rₜ is known, the following is obtained for the mean square displacement in the absence of external forces:

[symbol]

(r_{t+Δt}−rₜ)² [symbol] =2fD₀Δt, (1)

where f denotes the number of dimensions, D₀ denotes the diffusion coefficient and Δt denotes the time period that has elapsed between the measurement of the two positions r_{t+Δt} and rₜ. For a spherical particle, the diffusion coefficient D₀ is inversely proportional to the radius of the particle.

Below, the proportionality factor 2 f D₀ of the equation (1) is referred to generally as fluctuation intensity Φᵣ.

In addition to the translational movement, the impacts with the liquid molecules generate an erratic rotation of the particle.

If a magnetic particle, such as the detection particle from FIG. 1 or FIG. 2, moves through a liquid within a container, on the wall of which there is mounted a for example one-dimensional magnetic field sensor, for example Hall sensor, AMR or GMR magnetometer, MEMS sensor or SQUID, the measured magnetic field intensity will fluctuate over time. A cause for this is a changing distance between the particle and magnetic field sensor, and a change in the direction of the magnetic field owing to a rotation of the particle. From the measurement of the field intensity versus the time, it is possible, analogously to equation (1), to determine a fluctuation intensity Φ_{B}. The fluctuation intensity Φ_{B} is greater the smaller the particle is. In other words, the fluctuation intensity Φ_{B} will decrease if the magnetic detection particle is bound to a particle for detection, for example a cell for detection or the like, because the magnetic detection particle and the particle for detection move jointly through the sample or the liquid.

FIG. 4 is a schematic sectional illustration of a part of a microfluidic system 100 as per an exemplary embodiment. The microfluidic system 100 corresponds in this case to the microfluidic system from FIG. 1 or to a similar microfluidic system, wherein the illustration of FIG. 4 shows only a partial section of the microfluidic system in detail compared to FIG. 1. The microfluidic system 100 has, in the exemplary embodiment illustrated in FIG. 4, a polymer multi-layer structure.

Of the microfluidic system 100, FIG. 4 thus shows the magnetic field sensor 130, the microfluidic channel 140, a holding device 410 for holding the magnetic field sensor 130, a first polymer substrate 441 with the microfluidic channel 140, a second polymer substrate 442, a polymer membrane 444, and an aperture 445 or an aperture section 445 in the second polymer substrate 442.

The interface for coupling to the magnetic field sensor 130 is designed in this case as a part of the holding device 410, which is not explicitly illustrated in FIG. 4. Alternatively, the holding device 410 may represent the interface.

In other words, FIG. 4 shows a cross section through a multi-layer structure of the microfluidic system 100, composed of two polymer substrates 441 and 442, which are separated from one another by a flexible polymer membrane 444. The microfluidic channel 140 runs in the first polymer substrate 441. The aperture section 445 is formed in the second polymer substrate 442, such that the polymer membrane 444 is exposed in this region. The magnetic field sensor 130 is fastened to the holding device 410.

To carry out an analysis of a sample or for the detection of particles in a sample, for example in accordance with the method from FIG. 2 or a similar method, the holding device 410 is displaced in the direction of the multi-layer structure, in particular into the aperture section 445, until the magnetic field sensor 130 is in contact with the polymer membrane 444.

FIG. 5 shows a schematic sectional illustration of a part of a microfluidic system 100 as per an exemplary embodiment. Here, the microfluidic system 100 in FIG. 5 corresponds to the microfluidic system from FIG. 4, or the illustration in FIG. 5 corresponds to the illustration from FIG. 4 with the exception that the magnetic field sensor 130 fastened to the holding device 410 is, in FIG. 5, arranged in contact with, or moved into contact with, the polymer membrane 444.

In the exemplary embodiment illustrated in FIG. 5, the magnetic field sensor 130 is in this case pushed into the polymer membrane 444, such that the polymer membrane 444 is deformed. It can be achieved in this way that the magnetic field sensor 130 is in full contact with the polymer membrane 444, and is thus situated close to a sample when the latter is conducted through the microfluidic channel 140.

With reference to FIG. 1, FIG. 4 and FIG. 5, examples of variables, materials and dimensions will be discussed below.

An acquisition interval or measurement interval Δt may amount to for example 1 microsecond to 100 milliseconds, in particular 100 microseconds to 10 milliseconds. A measurement duration per particle may, in the case of exemplary embodiments without circulation or flow, amount to 10 seconds to 10 minutes, or may, in exemplary embodiments with circulation or flow, that is to say with a dwell time of the detection particle 106 in the detection region of the magnetic field sensor 130, amount to for example 500 milliseconds to 30 seconds. A field strength of a homogeneous magnetic field may amount to for example 10 microtesla to 1000 millitesla, in particular 500 microtesla to 500 millitesla. A diameter of the magnetic detection particles 106 may amount to for example 100 nanometers to 100 micrometers, in particular 500 nanometers to 10 micrometers.

Example materials for the polymer substrates 441 and 442 comprise polymers, in particular thermoplastics, for example PC, PP, PE, PMMA, COP, COC, wherein, for the polymer membrane 444, use may be made for example of elastomers, thermoplastic elastomers, thermoplastics, hotmelt adhesive films or the like.

A thickness of the polymer substrates 441 and 442 may amount to for example 0.1 millimeters to 10 millimeters, in particular 1 millimeter to 3 millimeters. A thickness of the polymer membrane 444 may amount to for example 5 micrometers to 500 micrometers, in particular 50 micrometers to 150 micrometers. Cross-sectional dimensions of the microfluidic channel 140 may amount to 10 by 10 square micrometers to 3 by 3 square millimeters, in particular 100 by 100 square micrometers to 1 by 1 square millimeters.

With reference to FIGS. 1 to 5, exemplary embodiments, fundamental principles and advantages will be summarized, and/or discussed in other words, below.

In the method 200 for detecting particles 104 or cells, magnetic detection particles 106 are used which, in the case of immunoassays, bind by immunoreaction specifically to cells or particles 104 for detection. After the detection particles 106 have been added into the sample 102, the magnetic field sensor 130 is utilized to determine the intensity and/or direction of the magnetic field versus the time of at least one detection particle 106 in at least one spatial dimension. From the recorded measurement signal 115, in the next step 220, the fluctuation intensity is ascertained. The fluctuation intensity for a detection particle 106 which is not bound to a cell or a particle 104 differs from that of a detection particle 106 which is bound to a cell or a particle 104. It is thus possible for the presence of the cells or particles 104 for detection to be determined by means of the fluctuation intensity.

In one exemplary embodiment, the microfluidic system 100 is designed as a polymer multi-layer structure which has an interface 110 to the for example single magnetic field sensor 130. The magnetic field sensor 130 may be arranged in or adjacent to an actuation unit for the lab-on-chip system or microfluidic system 100.

In one exemplary embodiment, the method for detecting has the following steps:

providing a sample 102 in a sample vessel;

providing a magnetic field sensor 130 in the vicinity of the sample vessel, in particular in contact with the wall of the sample vessel;

adding 240 magnetic detection particles 106, which are for example suspended in a liquid, wherein the detection particles 106 bind to the cells or particles 104 for detection, for example to antibodies;

measuring or acquiring the magnetic field intensity versus the time;

calculating or ascertaining 220 the fluctuation intensity Φ_{B}, as described for example with reference to FIG. 3; and

determining 230 the state of binding from the fluctuation intensity.

The concentration of the magnetic detection particles 106 is in this case, in the step 240 of adding, selected for example such that in each case only one single detection particle 106 is arranged within a range of the magnetic field sensor 130.

In one exemplary embodiment, in the method 200 for detecting, after the measurement of a detection particle 106 and identification of the state of binding thereof, the step 260 of circulating is performed, such that a circulation of the sample 102 is performed and another detection particle 106 can be measured. The circulation may be realized for example by means of an agitator or by means of external/internal pumping.

In a further exemplary embodiment, a three-dimensional magnetic field sensor 130 is used. It is thus possible for a rotational component of the fluctuation intensity to be at least partially separated from a translational component. Such an exemplary embodiment has the advantage in particular that the state of binding can be determined by means of two separate variables, that is to say rotational component and translational component, and thus even greater certainty in the detection of the state of binding can be achieved.

In another exemplary embodiment, the sample 102 is permeated, within the range of the magnetic field sensor 130 at least during the measurement process, by a homogeneous magnetic field generated in the step 250 of generating, for example by means of a Helmholtz coil arrangement. In this way, the magnetic moments of the detection particles 106 within the homogeneous magnetic field are aligned, and a rotational movement is prevented.

In one exemplary embodiment, in the step 240 of adding, use is made of paramagnetic detection particles 106. In this way, the magnetic moment of the detection particles 106 is formed in combination with the homogeneous magnetic field. This exemplary embodiment has the advantage in particular that the detection particles 106 in the sample 102 do not exhibit any attractive interaction with one another outside the homogeneous magnetic field, and therefore a coagulation of the detection particles 106 is prevented.

The fact that the rotational movement of the detection particles 106 is prevented in the ways stated above yields the advantage that the measured field strength H is only a function of the distance d to the magnetic field sensor 130. If this function is known, for example from corresponding simulations, it is possible from the measurement signal H(t) 115 to directly derive a trajectory d(t) of the detection particles 106. With the aid of equation (1), it is thus possible from the trajectory d(t) to calculate the diffusion coefficient D₀ and thus the radius of the detection particle 106. This has the advantage that a known variable, the radius of the free or unbound detection particle 106, is obtained as a measurement value, and it is thus for example possible to perform an exact and reliable calibration. It is expedient here to additionally take into consideration that the diffusion coefficient D₀ is not constant in the vicinity of a wall, but rather is dependent on the distance to the wall. The diffusion coefficient D₀ at the distance z from the wall can be determined from the trajectory d(t) if, for every spacing z, equation (1) is evaluated using the condition zₛₜₐᵣₜ=z. This has the advantage in particular that the diffusion coefficient D₀ and thus the radius can be determined even more exactly.

In a further exemplary embodiment, the magnetic field sensor 130 is arranged in contact with the microfluidic channel 140, and the sample 102 including the magnetic detection particles 106 is pumped through the microfluidic channel 140, for example by means of external or microfluidically integrated pumps. As soon as a detection particle 106 generates a measurement signal 115 at the magnetic field sensor 130, the flow is stopped, and the state of binding of the detection particle 106 is evaluated. The process is subsequently repeated for the next detection particle 106. This has the advantage in particular that the state of binding of each detection particle 106 can be determined exactly once. Furthermore, the detection particle 106 can be positioned exactly over the magnetic field sensor 130 such that a strong measurement signal 115 can be generated. This furthermore makes it possible to determine the number of detection particles 106 which have bound in each case to a cell for detection or to a particle 104 for detection and which have not. In this way, a concentration of the cells or particles 104 for detection can be estimated.

In a yet further exemplary embodiment, the magnetic field can, in the step 250 of generating, be generated such that, for the detection particle 106, a potential well is realized in the vicinity of the magnetic field sensor 130, that is to say in the manner of so-called magnetic tweezers. In this way, the detection particle 106 can be held even more exactly in the vicinity of the magnetic field sensor 130.

In one exemplary embodiment, the sample 102 is, in the step 260 of circulating, pumped continuously through the microfluidic channel 140, and the measurement of the detection particles 106 is performed as the flow passes the magnetic field sensor 130. In this case, it is advantageous if, before the evaluation of the measurement signal 115, low-frequency components in the measurement signal 115, which arise as a result of the variable distance to the magnetic field sensor 130 as flow passes by, are discarded. It is additionally expedient for the interval Δt between measurements to be kept as small as possible in order to minimize the component of the signal between two measurement points, which is caused by the constant relative movement between detection particle 106 and magnetic field sensor 130. To increase the amount of data recorded per detection particle 106, and thus the accuracy of the measurement, the liquid or sample 102 conducted past the magnetic field sensor 130 may be conducted past the magnetic field sensor 130 for at least a second time. It is thus possible to determine the probability that a detection particle 106 has bound to a cell for detection or to a particle 104 for detection. Using the known concentration of the detection particles 106, it is thus also possible to ascertain the concentration of the cells or particles 104 for detection.

Lab-on-chip systems or microfluidic systems 100 commonly have disposable parts which are used only for one measurement. For the implementation of the method 200 for detecting, it is therefore advantageous for the magnetic field sensor to be coupled removably to a disposable component, that is to say to offer an interface 110 to the magnetic field sensor 130, which may be arranged in an actuation unit for the lab-on-chip system. It is thus possible for costs for the lab-on-chip system or microfluidic system 100 to be reduced. It is furthermore possible to use more complex and more accurate magnetic field sensors 130.

The abovementioned immunoassay workflow composed of numerous steps can, in exemplary embodiments, be shortened. For example, all of the washing steps are eliminated, and it is only necessary for one substance, a buffer with functionalized magnetic detection particles 106, to be added to the sample 102. In this way, process times and material costs can be reduced. Furthermore, the shortened workflow can be more easily integrated into lab-on-chip systems or microfluidic systems 100.

By contrast to conventional detection methods, it is only necessary to use a small number of probes, such as for example magnetic detection particles 106 or fluorescence-marked molecules, to obtain a utilizable measurement signal 115. The method 200 for detecting makes it possible to determine the state of binding of an individual magnetic detection particle 106. In this way, it is possible to detect an individual cell or an individual particle 104.

The detection method of the method 200 for detecting, or using the detection device 120 and the microfluidic system 100, is contactless and requires no optically transparent access. Since magnetic fields permeate non-conductive substances generally without significant attenuation, there is a great amount of freedom in terms of the material selection.

In particular, it is possible for the method 200 for detecting to also be implemented through the skin of a patient for magnetic detection particles 106 in a blood vessel. It is thus possible for magnetic beads as detection particles 106 to be injected into a patient, for the state of binding thereof to be able to be continuously monitored, for example by means of a magnetic field sensor 130 on the wrist, which magnetic field sensor is for example integrated into a smartwatch or the like. This may be used for example for the early detection of cancer, pathogens, viruses or for the monitoring of the course of therapies.

By contrast to lab-on-chip systems with magnetic field sensors which are integrated directly into parts for single use, the interface 110 makes it possible for a magnetic field sensor 130, which is situated for example in the actuation system for the lab-on-chip system 100, to be positioned in the direct vicinity of the sample 102. Since the magnetic field sensor 130 can be utilized several times or in multiple microfluidic systems 100, costs for a lab-on-chip system or microfluidic system 100 can be lowered.

Where an exemplary embodiment comprises an “and/or” combination between a first feature and a second feature, this is to be read as meaning that the exemplary embodiment, in one embodiment, has both the first feature and the second feature and, in a further embodiment, has either only the first feature or only the second feature.

1. A method for detecting particles in a sample, comprising:
adding magnetic detection particles to the sample, the magnetic detection particles configured to bind to the particles for detection;
reading in a measurement signal from an interface to at least one magnetic field sensor, the measurement signal including at least one acquired characteristic of a magnetic field extending at least through a partial quantity of the sample;
ascertaining a fluctuation intensity of the at least one acquired characteristic of the magnetic field, the fluctuation intensity dependent on the detection particles; and
determining a state of binding of at least one detection particle as unbound or bound to a particle for detection based on the determined fluctuation intensity in order to detect the particles in the sample.

2. The method as claimed in claim 1, further comprising:
determining the state of binding as unbound when the fluctuation intensity lies in a first value range; and
determining the state of binding as bound to a particle for detection when the fluctuation intensity lies in a second value range, the second value range including lower fluctuation intensities than the first value range.

3. The method as claimed in claim 1, further comprising:
reading in the measurement signal to include at least one of an intensity and a direction of a magnetic field versus time.

4. The method as claimed in claim 1, further comprising:
adding a predefined quantity of the magnetic detection particles to the sample.

5. The method as claimed in claim 4, further comprising:
performing at least one of:
(i) adding magnetic detection particles that are configured to bind to at least one of antibodies and surface proteins of the particles for detection, wherein the particles for detection are biological particles; and
(ii) adding paramagnetic detection particles to the sample.

6. The method as claimed in claim 1, further comprising:
generating a magnetic field, the measurement signal read in including at least one acquired characteristic of the generated magnetic field.

7. The method as claimed in claim 6, further comprising:
generating at least one of (i) a homogeneous magnetic field as the magnetic field, and (ii) a magnetic field configured to immobilize at least one detection particle adjacent to the at least one magnetic field sensor.

8. The method as claimed in claim 1, further comprising:
circulating the sample with the added magnetic detection particles at least one of (i) before, (ii) during, and (iii) after a detection of the at least one acquired characteristic of the magnetic field.

9. A detection device comprising:
a reading-in module configured to read in a measurement signal from an interface;
an ascertainment module configured to ascertain using the measurement signal a fluctuation intensity of at least one acquired characteristic of a magnetic field represented by the measurement signal; and
a determination module configured to determine based on the fluctuation intensity a state of binding of at least one magnetic detection particle in the magnetic field as unbound or bound to a particle for detection,
wherein the detection device is configured to carry out a method to detect particles in a sample, the method including:
binding the magnetic detection particles to the particles for detection;
reading in the measurement signal from the interface to at least one magnetic field sensor using the reading-in module, the measurement signal including the at least one acquired characteristic of the magnetic field extending at least through a partial quantity of the sample;
ascertaining the fluctuation intensity of the at least one acquired characteristic of the magnetic field using the ascertainment module, the fluctuation intensity dependent on the detection particles; and
determining the state of binding of the at least one detection particle as unbound or bound to the particle for detection based on the determined fluctuation intensity in order to detect the particles in the sample using the determination module.

10. A microfluidic system for analyzing a sample, the microfluidic system comprising:
an interface configured to couple to at least one magnetic field sensor; and
a detection device configured to carry out a method to detect particles in the sample, wherein the method includes:
adding magnetic detection particles to the sample, the magnetic detection particles being configured to bind to the particles for detection;
reading in a measurement signal from the interface to at least one magnetic field sensor, the measurement signal including at least one acquired characteristic of a magnetic field extending at least through a partial quantity of the sample;
ascertaining a fluctuation intensity of the at least one acquired characteristic of the magnetic field, the fluctuation intensity dependent on the detection particles; and
determining a state of binding of at least one detection particle as unbound or bound to a particle for detection based on the determined fluctuation intensity in order to detect the particles in the sample,
wherein the measurement signal is configured to be transmitted via the interface to the detection device.

11. The microfluidic system as claimed in claim 10, further comprising:
a microfluidic channel in which the sample is configured to be conducted,
wherein at least one of the detection device and the interface are arranged adjacent to the microfluidic channel.

12. The microfluidic system as claimed in claim 11, wherein:
the interface is further configured to arrange the at least one magnetic field sensor in contact with, or move the at least one magnetic field sensor into contact with, a wall of the microfluidic channel.

13. The microfluidic system as claimed in claim 10, further comprising:
wherein the at least one magnetic field sensor is configured to be removably coupled or couplable to the interface.

14. The method as claimed in claim 1, wherein a computer program is configured to carry out the method.

15. The method as claimed in claim 14, wherein the computer program is stored on a machine-readable storage medium.
